(19) United States
(12) Patent Application Publication
(10) Pub. No.: US 2005/0085455 A1
(43) Pub. Date: Apr. 21, 2005

(54) Photodynamic therapy for local adipocyte reduction

(75) Inventor: James C. Chen, Bellevue, WA (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Light Sciences Corporation, Snoqualmie, WA (US)

(21) Appl. No.: 10/687,579

(22) Filed: Oct. 16, 2003

(51) Int. Cl. A61K 31/555; A61N 1/30

(52) U.S. Cl. 514/185; 514/410; 604/20

(57) ABSTRACT

The present invention is drawn to methods and compounds for transcutaneous photodynamic therapy (“PDT”) of target adipocyte cells or adipose tissue in a mammalian subject, which includes administering to the subject a therapeutically effective amount of a photosensitizing agent or a photosensitizing agent delivery system or a prodrug, where the photosensitizing agent or photosensitizing agent delivery system or prodrug selectively binds to the target tissue; and irradiating at least a portion of the subject with light at a wavelength absorbed by the photosensitizing agent or if prodrug, by a prodrug product thereof, where the light is provided by a light source, and where the irradiation is at low fluence rate that results in the activation of the photosensitizing agent or prodrug product. These methods of transcutaneous PDT are useful for the reduction of adipose tissue and adipocytes.

### TECHNICAL FIELD OF THE INVENTION

This invention relates generally to the field of medicine and pharmacotherapeutics with photosensitizing agents or other energy-activated agents. Specifically, provided herein are methods, compounds, compositions and kits useful for site specific delivery of a therapeutically effective amount of a photosensitizing agent to adipocytes. In particular, methods of using either an external or internal light source effective in providing transcutaneous photodynamic therapy for local adipocyte reduction are provided.

### BACKGROUND OF THE INVENTION

Obesity is a major public health problem that increases the risk of non-insulin-dependent diabetes mellitus, stroke, heart disease, liver disease, orthopedic disorders and some types of cancers. Obesity reflects increased adipocyte volume and increased adipocyte number. See Prins, J. et al., Biochem. Biophys. Research Comm. 201(2):500-507 (1994).

Obesity is typically treated by monitoring one's diet, exercise, and reducing the subcutaneous adipose layers by plastic surgery, liposuction, ultrasound and laser treatments. Due to the fast pace of modern society, many find it difficult to maintain a healthy diet and exercise regularly in order to prevent obesity.

Plastic surgery and liposuction are invasive procedures that require significant periods of recovery. Invasive procedures further subject the patient to risks of infection, bleeding, anesthesia risks and other post-surgical complications. Liposuction involves the introduction into the adipose layers of probes around 5 mm in diameter through holes in the skin to remove the adipose tissue. The disadvantages of liposuction include the creation of a visible lack of homogeneity in the form of depressions in the zone of insertion of the probe, excessive bleeding and nonselective removal of the cells of fat and stroma. See Paolini et al., U.S. Pat. No. 5,954,710. The disadvantage of utilizing subcutaneous ultrasonic probes also includes a visible lack of homogeneity. Paolini et al., U.S. Pat. No. 5,954,710, disclose the use of a laser for the removal of subcutaneous adipose layers. The laser device described comprises a needle for inserting and guiding the optical fiber emitting the laser beam in the adipose tissue to be treated. The disadvantage of using this device is that the treatment is invasive.

Clearly, there is a long-felt need for a method to treat obesity by reducing adipose tissue which method is noninvasive or minimally invasive and results in homogenous adipose tissue reduction. The present invention provides a device and a non-invasive or minimally invasive method for treating obesity involving the use of photodynamic therapy (PDT) to induce adipocyte reduction. This method and device are disclosed herein below.

### SUMMARY OF THE INVENTION

The present invention is based on the precise targeting of photosensitive agents or other energy activated agents, drugs and compounds to specific target cells or compositions of a subject or patient and to the method of activation of these targeted photosensitizer agents or other energy activated agents by subsequently administering to the subject light or ultrasonic energy at a relatively low intensity rate and over a prolonged period of time, utilizing a light or ultrasonic energy source that is either external or internal to the target tissues in order to achieve maximal cytotoxicity with minimal side effects.

One embodiment includes a method for photodynamic therapy (“PDT”) of subcutaneous adipose tissue in a mammalian subject comprising: administering to the subject a therapeutically effective amount of a photosensitizing agent or a photosensitizing agent delivery system or a prodrug, where the photosensitizing agent or photosensitizing agent delivery system or prodrug selectively binds to the target tissue which is an adipocyte. This step is followed by irradiating at least a portion of the subject with light at a wavelength or waveband absorbed by the photosensitizing agent or if a prodrug, by a prodrug product thereof, where the light is provided by a light source, and where the irradiation is at a relatively low fluence rate that results in the activation of the photosensitizing agent or prodrug product. In this embodiment, the photosensitizing agent or photosensitizing agent delivery system or prodrug is cleared from non-target tissues of the subject prior to irradiation.

Another embodiment includes a method for transcutaneous PDT of a target composition in a mammalian subject comprising: administering to the subject a therapeutically effective amount of a photosensitizing agent or a photosensitizing agent delivery system or a prodrug, where the photosensitizing agent or photosensitizing agent delivery system or prodrug selectively binds to the target composition. This step is followed by irradiating at least a portion of the subject with light at a wavelength or waveband absorbed by the photosensitizing agent or if a prodrug, by a prodrug product thereof, where said light is provided by a light source, and where the irradiation is at a relatively low fluence rate that results in the activation of the photosensitizing agent or said prodrug product. This embodiment contemplates that the photosensitizing agent or the photosensitizing agent delivery system or prodrug is cleared from non-target tissues of the subject prior to said irradiation. This embodiment also contemplates that light is delivered from a relatively low power noncoherent or coherent light source that is positioned in proximity to the adipose tissue, beneath the skin surface and external to the adipose tissue. Another embodiment includes a method of transcutaneous PDT of a target tissue in a mammalian subject as described above, where the light source is entirely external to the patient's intact skin layer.

Another embodiment is drawn to a method of transcutaneous PDT, where the photosensitizing agent is conjugated to a ligand. One embodiment includes a method of transcutaneous PDT, where the ligand is an antibody specific to adipocytes or an adipocyte component, such as lipoprotein lipase (see Sato et al., Poultry Science 78:1286-1291 (1999)). Other embodiments include methods of transcutaneous PDT, where the ligand is a peptide or polymer specific to adipocytes.

In certain embodiments drawn to a method of transcutaneous PDT, the photosensitizing agent is selected from the group consisting of: indocyanine green (ICG); methylene blue; toluidine blue; aminolevulinic acid (ALA); phthalocyanines; porphyrins; texaphyrins; chlorin compounds; purpurins; and any other agent that absorbs light in a range of 500 nm-1100 nm. More specifically, chlorin and purpurin compounds contemplated in certain embodiments include: mono-, di-, or polyamide aminodicarboxylic acid derivatives of cyclic or non-cyclic tetrapyrroles (see Bommer et al., U.S. Pat. Nos. 4,675,338 and 4,693,885, each of which is hereby incorporated in its entirety herein); and alkyl ether derivatives of pyropheophorbide-a with N-substituted cyclic imides (purpurin-18 imides) (see Pandey et al., WO 99/67249). Another embodiment contemplates that the photosensitizing agent is mono-L-aspartyl chlorin e⁶ (NPe⁶).

Another embodiment includes a method of transcutaneous PDT, where the activation of the photosensitizing agent will likely occur within 30 minutes to 72 hours of irradiation, more preferably within 60 minutes to 48 hours of irradiation and most preferably within 3 hours to 24 hours of irradiation. Of course, clinical testing will be required to determine the optimal illumination time. In addition, it is contemplated that the total fluence delivered will preferably be between 30 Joules to 25,000 Joules, more preferably be between 100 Joules and 20,000 Joules, and most preferably be between 500 Joules to 10,000 Joules. Clinical testing will determine the optimal total fluence required to reduce the adipose tissue.

A further embodiment is drawn to a method for transcutaneous photodynamic therapy of target tissue in a mammalian subject comprising: administering to the subject a therapeutically effective amount of a first conjugate comprising a first member of a ligand-receptor binding pair conjugated to an antibody or antibody fragment, where the antibody or antibody fragment selectively binds to a target antigen found on adipocytes. This step is followed by administering to the subject a therapeutically effective amount of a second conjugate comprising a second member of the ligand-receptor binding pair conjugated to a photosensitizing agent or photosensitizing agent delivery system or prodrug, where the first member binds to the second member of the ligand-receptor binding pair. A subsequent step includes irradiating at least a portion of the subject with light at a wavelength or waveband absorbed by the photosensitizing agent or if prodrug, by the product thereof. This embodiment further includes that the light is provided by a light source and that the irradiation is at a relatively low fluence rate that results in the activation of the photosensitizing agent or prodrug product.

Still further embodiments are drawn to methods of transcutaneous PDT where the ligand-receptor binding pair is selected from the group consisting of: biotin-streptavidin and antigen-antibody. A further embodiment is drawn to the presently disclosed methods where the antigens are adipocyte antigens and the ligand-receptor binding pair includes biotin-streptavidin. In this embodiment, the activation of photosensitizer agents by a relatively low fluence rate light source over a prolonged period of time results in the destruction or reduction of the adipocytes.

Another embodiment contemplates a transcutaneous PDT method where the photosensitizing agent delivery system comprises a liposome delivery system consisting essentially of the photosensitizing agent.

Yet another embodiment includes a method for transcutaneous ultrasonic therapy of a target tissue in a mammalian subject comprising: administering to the subject a therapeutically effective amount of an ultrasonic sensitizing agent or an ultrasonic sensitizing agent delivery system or a prodrug, where the ultrasonic sensitizing agent or ultrasonic sensitizing agent delivery system or prodrug selectively binds to adipocytes. This step is followed by irradiating at least a portion of the subject with ultrasonic energy at a frequency that activates the ultrasonic sensitizing agent or if a prodrug, by a prodrug product thereof, where the ultrasonic energy is provided by an ultrasonic energy-emitting source. This embodiment further provides that the ultrasonic therapy drug is cleared from non-target tissues of the subject prior to irradiation. This embodiment includes a method for transcutaneous ultrasonic therapy of a target tissue, where the target tissue is adipose tissue.

Other certain embodiments contemplate that the ultrasonic energy-emitting source is external to the patient's intact skin layer or is inserted underneath the patient's intact skin layer. An additional embodiment provides that the ultrasonic sensitizing agent is conjugated to a ligand and more preferably, where the ligand is selected from the group consisting of: an adipocyte specific antibody, an adipocyte specific peptide and an adipocyte specific polymer. Other embodiments contemplate that the ultrasonic sensitizing agent is selected from the group consisting of: indocyanine green (ICG); methylene blue; toluidine blue; aminolevulinic acid (ALA); phthalocyanines; porphyrins; texaphyrins; pyropheophorbide compounds; chlorin compounds; purpurins; and any other agent that absorbs light in a range of 500 nm-1100 nm. More specifically, chlorin and purpurin compounds contemplated, include: mono-, di-, or polyamide aminodicarboxylic acid derivatives of cyclic or non-cyclic tetrapyrroles (see Bommer et al., U.S. Pat. Nos. 4,675,338 and 4,693,885); and alkyl ether derivatives of pyropheophorbide-a with N-substituted cyclic imides (purpurin-18 imides) (see Pandey et al., WO 99/67249). An embodiment contemplates that the photosensitizing agent is mono-L-aspartyl chlorin e⁶ (NPe⁶).

Other embodiments include the presently disclosed methods of transcutaneous PDT, where the light source is positioned in proximity to the target tissue of the subject and is selected from the group consisting of: an LED light source; an electroluminesent light source; an incandescent light source; a cold cathode fluorescent light source; organic polymer light source; and inorganic light source. An embodiment includes the use of an LED light source.

Yet other embodiments of the presently disclosed methods are drawn to use of light of a wavelength that is from about 500 nm to about 1100 nm, preferably greater than about 650 nm and more preferably greater than about 700 nm. An embodiment of the present method is drawn to the use of light that results in a single photon absorption mode by the photosensitizing agent.

Additional embodiments include compositions of photosensitizer-targeted delivery systems comprising: a photosensitizing agent and a ligand that binds a receptor on the target tissue with specificity. In one embodiment the photosensitizing agent of the targeted delivery system is conjugated to the ligand that binds a receptor on the target lesion with specificity. Preferably, the ligand comprises an antibody that binds to a receptor and the receptor is an antigen on adipocytes. Even further preferred is lipoprotein lipase antigen, which binds specifically and preferentially to lipoprotein lipase monoclonal antibodies (see Sato et al., Poultry Science 78:1286-1291 (1999)).

A further embodiment contemplates that the photosensitizing agent is selected from the group consisting of: indocyanine green (ICG); methylene blue; toluidine blue; aminolevulinic acid (ALA); phthalocyanines; porphyrins; texaphyrins; chlorin compounds; purpurins; and any other agent that absorbs light in a range of 500 nm-1100 nm. Another embodiment of this invention contemplates that the photosensitizing agent is mono-L-aspartyl chlorin e⁶ (NPe⁶).

Still another embodiment includes that the ligand-receptor binding pair is selected from the group consisting of: biotin-streptavidin and antigen-antibody.

Yet another embodiment contemplates that the photosensitizing agent comprises a prodrug.

Other embodiments contemplate methods for transcutaneous PDT to destroy a target cell in a mammalian subject comprising: administering to the subject a therapeutically effective amount of a photosensitizing agent or a photosensitizing agent delivery system or a prodrug, where the photosensitizing agent or photosensitizing agent delivery system or prodrug selectively binds to the target cell. This step is followed by irradiating at least a portion of the subject with light at a wavelength or waveband absorbed by the photosensitizing agent or if prodrug, by a prodrug product thereof, where the light is provided by a light source, and where the irradiation is at a relatively low fluence rate that results in the activation of the photosensitizing agent or prodrug product and the destruction of the target cell. This embodiment contemplates that the photosensitizing agent is cleared from non-target tissues of the subject prior to said irradiation.

Still a further embodiment provides that a photosensitizing agent is delivered locally or regionally by administration of a drug delivery patch method. This embodiment also provides for the use of ultrasound to drive and direct the photosensitizing agent into the subcutaneous fatty tissues. An alternative methodology provides for the injection percutaneously into the treatment site.

### BRIEF DESCRIPTION OF THE DRAWINGS

FIG. 1 shows a diagram that demonstrates transcutaneous PDT using a laser diode light source that is focused (3) and non-focused and placed at an angle (2) to the adipose tissue (5) and that is external to the skin layer (4).

FIG. 2 shows PDT using an optical fiber (6) delivery of light from a laser diode light source (2) that is inserted underneath the skin layer (4), but external to the outer membrane of the adipocyte (5).

FIG. 3 shows transcutaneous PDT using a light source that is comprised of multiple LEDs arrayed in a strip (7) or a fiber optic diffuser (7) and placed external to the skin layer (4).

FIG. 4 demonstrates transcutaneous PDT using an optical diffuser (8) attached to an optical fiber with delivery of light from a laser diode light source (not shown). FIG. 4A shows an end on view of the optical fiber with a mirrored surface (9) directing light toward the treatment area.

### DETAILED DESCRIPTION OF THE INVENTION

Apoptosis is a specific form of cell death. Apoptosis occurs under normal conditions such as during embryogenesis and physiological involution of adult tissue. It also occurs during abnormal conditions or may be induced by exposure to radiation, neoplastic drugs and other toxins. It has been suggested that apoptosis may play a role in adipocyte reduction. See Prins, J. et al., Diabetes 46:1939-1944 (1997)), and Prins, J. et al., Biochem. Biophys. Research Comm. 205(1):625-630 (1994).

One form of energy-activated therapy is photodynamic therapy (PDT). PDT has been applied to treat a range of diseases including cancer and heart disease. See Oleinick, N. et al., Radiation Research 150: S146-S156 (1998). PDT may be used to induce apoptosis. See Ahmad, N. et al., Proc. Natl. Acad. Sci. 95:6977-6982 (1998); and Kessel, D. et al., Cell Death and Differentiation 6:28-35 (1999).

PDT is performed by first administering a photosensitive compound systemically or topically, followed by illumination of the treatment site at a wavelength or waveband which closely matches the absorption spectra of the photosensitizer. In doing so, singlet oxygen and other reactive species are generated leading to a number of biological effects resulting in cytotoxicity. The depth and volume of the cytotoxic effect in tissue depends on the complex interactions of light penetration in tissue, the photosensitizer concentration and cellular location, and availability of molecular oxygen.

A large number of PDT light sources and methods of use have been described. However, reports describing the sources and effects of transcutaneous light delivery for PDT purposes are more limited. It has generally been accepted that the ability of a light source external to the body to cause clinically useful cytotoxicity is limited in depth to a range of 1-2 cm or less depending on the photosensitizer. Thus, gradually reduction of subcutaneous adipose tissue may occur in a noninvasive manner without causing extensive damage to deep tissue.

The methods, compounds, compositions and kits disclosed herein provide that PDT be used to induce apoptosis rather than necrosis of adipocytes. By administering a therapeutically effective concentration of photosensitizer or energy activated agent and modulating the amount of irradiating energy, the degree of necrosis and subsequent inflammation can be minimized. Further, this will ensure that other adverse side effects due to rapid triglyceride mobilization can be avoided or lessened. The apoptotic process enables a much more controlled reduction of the deposits of fatty tissue compared to a process in which such tissue is reduced by an induction of cellular necrosis.

However, treatment of subcutaneous adipose layers in this manner may be associated with inadvertent skin damage due to accumulation of the photosensitizer in the skin which is a property of all systemically administered sensitizers in clinical use. For example, clinically useful porphyrins such as Photophrin® (QLT, Ltd. brand of sodium porfimer) are associated with photosensitivity lasting up to 6 weeks. Purlytin®, which is a purpurin, and Foscan®, which is a chlorin, sensitize the skin for several weeks. Indeed, efforts have been made to develop photoprotectants to reduce skin photosensitivity (see Dillon et al., Photochemistry and Photobiology 48(2):235-238 (1988); and Sigdestad et al., British J. of Cancer 74:S89-S92 (1996)). In fact, PDT protocols involving systemic administration of photosensitizer require that the patient avoid sunlight and bright indoor light to reduce the chance of skin phototoxic reactions.

One PDT modality discloses the use of an intense laser source to activate drug within a precisely defined boundary. See Fisher et al., U.S. Pat. No. 5,829,448. A two-photon methodology requires a high power laser for drug activation with a highly collimated beam that requires a high degree of spatial control. This type of treatment is not practical for treating large areas of adipose tissue since the beam would have to be swept across the skin surface in some sort of set, repeatable pattern over time. Patient or organ movement would be a problem, because the beam could become misaligned. Non-target tissue or skin and subcutaneous tissue photosensitivity is not addressed in the literature available. Any photosensitizer in the path of the beam would be activated and cause unwanted collateral tissue damage.

Therefore, a one-photon method is preferred in the PDT reduction of adipose tissue. The one-photon method allows for a prolonged exposure at a lower fluence rate, which promotes the protection of non-target tissue or skin and subcutaneous normal tissue and reduces collateral tissue damage.

This invention further discloses the selective binding of the photosensitizing agent to specific target tissue antigens, such as those found on the surface of or within adipocytes. This targeting scheme decreases the amount of sensitizing drug required for effective therapy, which in turn reduces the total fluence, and the fluence rate needed for effective photoactivation.

A light source far less intense than a high powered laser and brief exposure using collimated light as disclosed by W. G. Fisher et al., in Photochemistry and Photobiology 66(2):141-155 (1997), is preferred. The present invention allows for the use of a low power non-coherent light source utilized for longer than about 1 hour to increase photoactivation depth.

This invention provides methods and compositions for treating a target tissue or destroying or impairing a target cell or composition in a mammalian subject by the specific and selective binding to the target tissue, cell or composition of a photosensitizer agent. This method comprises irradiating at least a portion of the subject with light at a wavelength absorbed by said photosensitizing agent that under conditions of activation during photodynamic therapy using a relatively low fluence rate, but an overall high total fluence dose results in minimal collateral tissue damage.

Terms as used herein are based upon their art recognized meaning and from the present disclosure should be clearly understood by the ordinary skilled artisan. For sake of clarity, terms may also have particular meaning as would be clear from their use in context. For example, transcutaneous more specifically herein refers to the passage of light through unbroken tissue. Where the tissue layer is skin or dermis, transcutaneous includes transdermal and the light source is external to the outer skin layer. However, where transillumination refers herein to the passage of light through a tissue layer, such as a layer of adipose tissue, the light source is external to the adipose tissue, but internal or implanted into the subject or patient.

Specifically, the present embodiments are based on the precise targeting of photosensitive agents or drugs and compounds to specific target antigens of a subject or patient and to the method of activation of targeted photosensitizer agents by subsequently administering to the subject light of a relatively low fluence rate over a prolonged period of time from a light source that is external to the target tissue in order to achieve maximal cytotoxicity or reduction of adipocytes over time with minimal side effects or collateral tissue damage.

Further, as used herein “target cells” or “target tissues” are those cells or tissues, respectively, that are intended to be impaired or destroyed by this treatment method. Target cells or target tissues take up the photosensitizing agent; then when sufficient radiation is applied, these cells or tissues are impaired or destroyed. Target cells are those cells in target tissues, which include, but are not limited to, adipocytes and preadipocytes.

“Non-target cells” are all the cells of an intact animal that are not intended to be impaired or destroyed by the treatment method. These non-target cells include but are not limited to stroma cells, and other normal tissue, not otherwise identified to be targeted.

“Destroy” is used to mean kill the desired target cell. “Impair” means to change the target cell in such a way as to interfere with its function. For example, North et al. observed that after exposure to light of benzoporphyrin derivatives (“BPD”)-treated, virus-infected T cells, holes developed in the T cell membrane, which increased in size until the membrane completely decomposed (Blood Cells 18:129-40 (1992)). Target cells are understood to be impaired or destroyed even if the target cells are ultimately disposed of by macrophages.

“Photosensitizing agent” is a chemical compound which homes to one or more types of selected target cells and, when contacted by radiation, absorbs the light, which results in impairment or destruction of the target cells. Virtually any chemical compound that homes to a selected target and absorbs light may be used in this invention. Preferably, the chemical compound is nontoxic to the animal to which it is administered or is capable of being formulated in a nontoxic composition. Preferably, the chemical compound in its photodegraded form is also nontoxic. A comprehensive listing of photosensitive chemicals may be found in Kreimer-Birnbaum, Sem. Hematol. 26:157-73 (1989). Photosensitive compounds include, but are not limited to: indocyanine green (ICG); methylene blue; toluidine blue; aminolevulinic acid (ALA); phthalocyanines; porphyrins; texaphyrins; bacteriochlorins, merocyanines, psoralens, benzoporphyrin derivatives (BPD) and porfimer sodium and pro-drugs such as δ-aminolevulinic acid, which can produce drugs such as protoporphyrin. Also, included are: chlorin compounds, purpurins, and any other agent that absorbs light in a range of 500 nm-1100 nm. More specifically, chlorin and purpurin compounds contemplated in the present invention, include: mono-, di-, or polyamide aminodicarboxylic acid derivatives of cyclic or non-cyclic tetrapyrroles (see Bommer et al., U.S. Pat. Nos. 4,675,338 and 4,693,885); and alkyl ether derivatives of pyropheophorbide-a with N-substituted cyclic imides (purpurin-18 imides) (see Pandey et al., WO 99/67249). Specifically, included are derivatives of mono-L-aspartyl chlorin e6 (NPe⁶) and any other agent that absorbs light in a range of 500 nm-1100 nm.

“Radiation” as used herein includes all wavelengths. Preferably, the radiation wavelength is selected to match the wavelength(s) that excites the photosensitive compound. Even more preferably, the radiation wavelength matches the excitation wavelength of the photosensitive compound and has low absorption by the non-target cells and the rest of the intact animal, including blood proteins. For example, the preferred wavelength for NPe⁶ is the convenient range of 600 to 800 nanometers, with the preferred compounds absorbing in the 620-760 nanometer range.

The radiation is further defined by its intensity, duration, and timing with respect to dosing with the photosensitive agent. The intensity or fluence rate must be sufficient for the radiation to penetrate skin and reach the target cells, target tissues or target compositions. The duration or total fluence dose must be sufficient to photoactivate enough photosensitive agent to act on the target cells. Both intensity and duration must be limited to avoid overtreating the animal. Timing with respect to dosing with the photosensitive agent is important, because (1) the administered photosensitive agent requires some time to home in on target cells and (2) the blood level of many photosensitive agents decreases rapidly with time.

This invention provides a method of treating an animal, which includes, but is not limited to, humans and other mammals. The term “mammals” or “mammalian subject” also includes farm animals, such as cows, hogs and sheep, as well as pet or sport animals such as horses, dogs and cats.

By “intact animal” is meant that the whole, undivided animal is available to be exposed to radiation. No part of the animal is removed for separate radiation. The entire animal need not be exposed to radiation. Only a portion of the intact animal subject may or need be exposed to radiation.

“Transcutaneously” is used herein as meaning through the skin of an animal subject.

Briefly, the photosensitizing agent is generally administered to the animal before the animal is subjected to radiation.

Preferred photosensitizing agents include, but are not limited to: indocyanine green (ICG) (for example, see WO 92/00106 (Raven et al.); WO97/31582 (Abels et al.) and Devoisselle et al., SPIE 2627:100-108 (1995)); methylene blue; toluidine blue; and pro-drugs such as delta-aminolevulinic acid, which can produce drugs such as protoporphyrin; bacteriochlorins; phthalocyanines; porphyrins; texaphyrins; chlorin compounds; purpurins; merocyanines; psoralens, and any other agent that absorbs light in a range of 500 nm-1100 nm. More specifically, chlorin and purpurin compounds contemplated in the present invention, include: mono-, di-, or polyamide aminodicarboxylic acid derivatives of cyclic or non-cyclic tetrapyrroles (see Bommer et al., U.S. Pat. Nos. 4,675,338 and 4,693,885); and alkyl ether derivatives of pyropheophorbide-a with N-substituted cyclic imides (purpurin-18 imides) (see Pandey et al., WO 99/67249). A further photosensitizing agent is mono-L-aspartyl chlorin e6 (NPe6) (see U.S. Pat. No. 4,693,885).

The photosensitizing agent is administered locally or systemically. The photosensitizing agent is administered orally or by injection, which may be intravenous, subcutaneous, intramuscular or intraperitoneal. The photosensitizing agent also can be administered externally or topically via patches or implants.

The photosensitizing agent also can be conjugated to specific ligands reactive with a target, such as receptor-specific ligands or immunoglobulins or immunospecific portions of immunoglobulins, permitting them to be more concentrated in a desired target cell or microorganism. The photosensitizing agent may be further conjugated to a ligand-receptor binding pair, which includes, but is not limited to, biotin-streptavidin and antigen-antibody. This conjugation may permit lowering of the required dose level since the material is more selectively targeted and less is wasted in distribution into other tissues whose destruction must be avoided.

The photosensitizing agent, in one embodiment, can be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel, Introduction to Pharmaceutical Dosage Forms, Fourth Edition, p. 126, 1985). The photosensitizing agent can be administered in a dry formulation, such as tablets, pills, capsules, powders, granules, suppositories or patches. The photosensitizing agent also may be administered in a liquid formulation, either alone with water, or with pharmaceutically acceptable excipients, such as are disclosed in Remington's Pharmaceutical Sciences, Mack Publishing Company, Easton, Pa., 15th Edition, 1975. The liquid formulation also can be a suspension or an emulsion. In particular, liposomal or lipophilic formulations are most desirable. If suspensions or emulsions are utilized, suitable excipients include water, saline, dextrose, glycerol, and the like. These compositions may contain minor amounts of nontoxic auxiliary substances such as wetting or emulsifying agents, antioxidants, pH buffering agents, and the like.

The dose of photosensitizing agent will vary with the target cell(s) sought, the optimal blood level (see Example 1), the animal's weight and the timing of the radiation. Depending on the photosensitizing agent used, an equivalent optimal therapeutic level will have to be established. Preferably, the dose is calculated to obtain a blood level between about 0.001 and 100 μg/ml. Preferably, the dose will obtain a blood level between about 0.01 and 10 μg/ml.

This method comprises irradiating at least a portion of the subject with light at a wavelength or waveband absorbed by said photosensitizing agent that under conditions of activation during photodynamic therapy using a relatively low fluence rate, but also at an overall high total fluence dose resulting in minimal collateral tissue damage. It is contemplated that the optimal total fluence will be determined clinically using a light dose escalation trial. It is further contemplated that the total fluence will preferably be in the range of 30 to 25,000 Joules/cm², and more preferably be in the range from 100 to 20,000 Joules/cm², and most preferably be in the range from 500 to 10,000 Joules/cm².

The methods comprise irradiating at least a portion of the subject with light at a wavelength or waveband absorbed by said photosensitizing agent that under conditions of activation during photodynamic therapy using a relatively low fluence rate, but an overall high total fluence dose resulting in minimal collateral normal tissue damage. What is meant by “relatively low fluence rate” is a fluence rate that is lower than that typically used and one that generally does not result in significant damage to collateral or non-target tissues. Specifically, the intensity of radiation used to treat the target cell or target tissue is preferably between about 5 and 100 mW/cm². More preferably, the intensity of radiation is between about 10 and 75 mW/cm². Most preferably, the intensity of radiation is between about 15 and 50 mW/cm².

The duration of radiation exposure is preferably between about 30 minutes and 72 hours. More preferably, the duration of radiation exposure is between about 60 minutes and 48 hours. Most preferably, the duration of radiation exposure is between about 2 hours and 24 hours. Of course, routine clinical testing will be useful to determine the optimal fluence rate and total fluence delivered to the treatment site.

While not wishing to be limited by a theory, it is proposed herein that a photosensitizer agent can be substantially and selectively photoactivated in the target cells and target tissues within a therapeutically reasonable period of time and without excess toxicity or collateral damage to non-target tissues. Thus, there appears to be a therapeutic window bounded by the photosensitizer agent dosage and radiation dosage. The formation of photodegradation products of a photosensitizer agent was used as an indicator of photoactivation. Photoactivation of a photosensitizer agent has been postulated to cause the formation of singlet oxygen, which has a cytotoxic or apoptotic effect.

Additionally, certain embodiments are drawn to methods for transcutaneous ultrasonic therapy of adipose tissue in a mammalian subject or patient by first administering to the subject a therapeutically effective amount of a first conjugate comprising a first member of a ligand-receptor binding pair conjugated to an antibody or antibody fragment, wherein said antibody or antibody fragment selectively binds to a target antigen of adipocytes; and simultaneously or subsequently administering to the subject a therapeutically effective amount of a second conjugate comprising a second member of the ligand-receptor binding pair conjugated to an ultrasonic sensitizing agent or ultrasonic sensitizing agent delivery system or prodrug, wherein the first member binds to the second member of the ligand-receptor binding pair. These steps are followed by irradiating at least a portion of the subject with energy at a wavelength absorbed by said ultrasonic sensitizing agent or if ultrasonic sensitizing agent delivery system, by the product thereof, wherein said energy is provided by an energy source that is external to the subject; and wherein said ultrasound is at a relatively low intensity rate that results in the activation of said ultrasonic sensitizing agent or prodrug product.

While one embodiment is drawn to the use of light energy in a photodynamic therapy of adipose tissue using light and photosensitizer agents, other forms of energy are within the scope of this invention and understandable by one of ordinary skill in the art. Such forms of energy include, but are not limited to: thermal, sonic, ultrasonic; chemical; photo or light; microwave; ionizing, such as: x-ray, and gamma ray; and electrical. For example, sonodynamically induced or activated agents include, but are not limited to: gallium-porphyrin complex; and other porphyrin complexes, such as protoporphyrin and hematoporphyrin. See Yumita et al., Cancer Letters, 112: 79-86, 1997; and Umemura et al., Ultrasonics Sonochemistry 3:S187-S191 (1996). This embodiment further contemplates the use of an energy source that is external to the target tissue. The target tissues may include and may relate to adipocytes, per se.

The ordinary skilled artisan would be familiar with various ligand-receptor binding pairs, including those known and those currently yet to be discovered. Those known, include, but are not limited to the group consisting of: biotin-streptavidin and antigen-antibody. This invention contemplates an embodiment that includes the use of biotin-streptavidin as the ligand-receptor binding pair. However, the ordinary skilled artisan would readily understand from the present disclosure that any ligand-receptor binding pair may be useful provided the ligand-receptor binding pair demonstrate a specificity for the binding by the ligand to the receptor and further provided that the ligand-receptor binding pair permit the creation of a first conjugate comprising a first member of the ligand-receptor binding pair conjugated to an antibody or antibody fragment, wherein said antibody or antibody fragment selectively binds to a target antigen of adipocytes; and further permit the creation of a second conjugate comprising a second member of the ligand-receptor binding pair conjugated to an energysensitizing or photosensitizing agent or energysensitizing or photosensitizing agent delivery system or prodrug, and further wherein the first member binds to the second member of the ligand-receptor binding pair.

Another group of ligand receptor pairs includes the conjugation of an energysensitizing or photosensitizing agent or energysensitizing or photosensitizing agent delivery system or prodrug to a first member of the ligand-receptor binding pair selected from the group consisting of antibody to an adipocyte specific antigen; a ligand bindable to a specific adipocyte cell receptor; and other ligands bindable to specific adipocyte cellular surface components. Such first ligand-receptor member pair will selectively and specifically bind to the second member of the ligand-receptor binding pair, which may be an adipocyte specific antigen, adipocyte specific receptor or other adipocyte specific cellular surface component. In this manner, an energy-activating agent is specifically delivered to its adipocyte target cell corresponding to the ligand-receptor binding pair selected. For example, monoclonal antibody directed against lipoprotein lipase antigen binds specifically and preferentially to lipoprotein lipase (see, Sato et al., Poultry Science 78:1286-1291 (1999)).

Another embodiment is drawn to a method where the photosensitizing agent delivery system includes a liposome delivery system consisting essentially of the photosensitizing agent, however the ordinary skilled artisan would readily understand from the present disclosure that other delivery systems may be used. In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. A still further embodiment contemplates the disclosed method where the photosensitizing agent delivery system utilizes both a liposome delivery system and a photosensitizing agent, where each is separately conjugated to a second member of the ligand-receptor binding pair, and where the first member binds to the second member of the ligand-receptor binding pair, and more preferably where the ligand-receptor binding pair is biotin-streptavidin. This embodiment further contemplates that the photosensitizing agent as well as the photosensitizing agent delivery system may both be specifically targeted through the selective binding to a target tissue antigen by the antibody or antibody fragment of the first member binding pair. Such dual targeting is envisioned to enhance the specificity of uptake and to increase the quantity of uptake.

Having now generally described the invention, the same will be more readily understood through reference to the following examples, which are provided by way of illustration, and are not intended to be limiting of the present invention, unless specified.

### EXAMPLE 1

### Transcutaneous Photodynamic Therapy of Adipose Tissue

The photosensitizer may be administered systemically or regionally. In the case of systemic administration, the photosensitizer is conjugated to an agent that enables selective uptake of by the adipose tissue or adipocytes. In the case of regional delivery, the photosensitizer may be administered topically. Topical administration may be followed by a method, such as ultrasound, which enhances skin permeation and localization into the subcutaneous adipose tissue. Alternatively, the photosensitizer may be injected percutaneously into the treatment site where diffusion occurs and enables proper dispersal of the photosensitizer.

The photoactivation process that is preferred is one that induces apoptosis and not necrosis of adipocytes. This reduces inflammation and other side effects due to rapid triglyceride mobilization. The apoptotic process enables a controlled reduction of the adipose tissue as compared to a process whereby necrosis occurs. The triglycerides within the adipocytes subjected to PDT are gradually liberated and metabolized by the surrounding cells.

Apoptosis may be determined in tissue explants by observing characteristic “laddering” following gel electrophoresis, which confirms the occurrence of specific endonuclease-induced DNA cleavage, chromatin clumping, and lipid-filled interstitial macrophages.

A. Adipocytes and adipose tissue may be effectively decreased by transcutaneous photodynamic therapy. A targeted antibody-photosensitizer conjugate (APC) is prepared by linking a photosensitizer agent, such as NPe⁶ to a monoclonal antibody binding to an adipocyte specific antigen, such as lipoprotein lipase. This APC is delivered to the treatment site by any number of means available to the skilled artisan. For example, the APC may be delivered by injection locally underneath the subcutaneous skin layer or systemically by intravenous injection. The delivery of other formulations of APC may include: oral or topical formulations.

Elstrom et al., U.S. Pat. No. 5,999,847, teach the use of localized and transient pressure waves that are applied to tissue adjacent to target cells by means of a light source and a coupling interface placed in contact with the tissue that converts light from the light source into acoustic energy. The pressure waves cause transient poration of the cell membranes. Therapeutic agents are delivered to the site of the localized pressure waves by any suitable means, such as by injection with a needle. The light source and coupling interface can be incorporated into a catheter for application of the pressure waves to diseased blood vessels. A manually manipulable surgical device incorporating a needle for injecting the agent, light source, and coupling interface may also be used.

Excess photosensitizer conjugates are naturally eliminated from the body. One or more light sources are strategically placed or implanted near the tissue to be treated. Following a sufficient amount of time to permit clearing of the conjugates from the non-target tissues, such as 6 hours, the light sources are activated, irradiating the target tissue with at relatively low fluence rate, such as 50 mW/cm² for 5 hours but resulting in a high total fluence dose of light, such as 900 Joules/cm², in the wavelength from about 620 nm to about 760 nm. The light may be applied internally or externally.

The specific dose of photosensitizer conjugate is that which results in a concentration of active NPe⁶ sufficient to obtain a blood level between about 0.001 and 100 μg/ml. and more preferably, a dose of between about 0.01 and 10 μg/ml. However, it is well within the skill of the ordinary skilled artisan to determine the specific therapeutically effective dose using standard clinical practices and procedures.

Similarly, the specific fluence rate and total fluence dose may be routinely determined from the disclosure herein.

Additionally, the conjugate above could be further conjugated to an imaging agent such as technetium. Thus, the method could further comprise the steps of performing a nuclear medicine scan and imaging the sites to be treated.

B. Alternatively, following the disclosure of Example 1A, a second APC may be constructed by linking a photosensitizer agent that binds selectively to a second antigen, other than lipoprotein lipase and which also is primarily present or associated on adipocytes. The photosensitizer could instead be linked to a receptor-ligand binding pair, where one of the binding pair is specifically associated with adipocytes and the other of the binding pair is linked to the photosensitizer agent. Such receptor ligand binding pairs could include: hormone-hormone receptor; chemokine-chemokine receptor; or other signal transduction receptor and its natural ligand. The ligand-receptor binding pair or APC is infused intravenously and is taken up in the adipose tissue. When unbound, APC is eliminated from the body. Internal or external light sources may be used to activate the targeted drug, however, in this Example an external light source is contemplated.

Any number of antigens or ligand binding pair components may be selected, provided that the component is specifically associated with adipocytes. Such antigens or ligand binding pair components would be known to those skilled in the art. The selection of a specific photosensitizer agent may be made, provided the photosensitizer agent is activated by a light wavelength of from 500 nm to 1100 nm, and more preferably a wavelength of 620 nm, and most preferably by a wavelength of 700 nm or greater. Such photosensitizer agents as provided in this disclosure are contemplated for use herein.

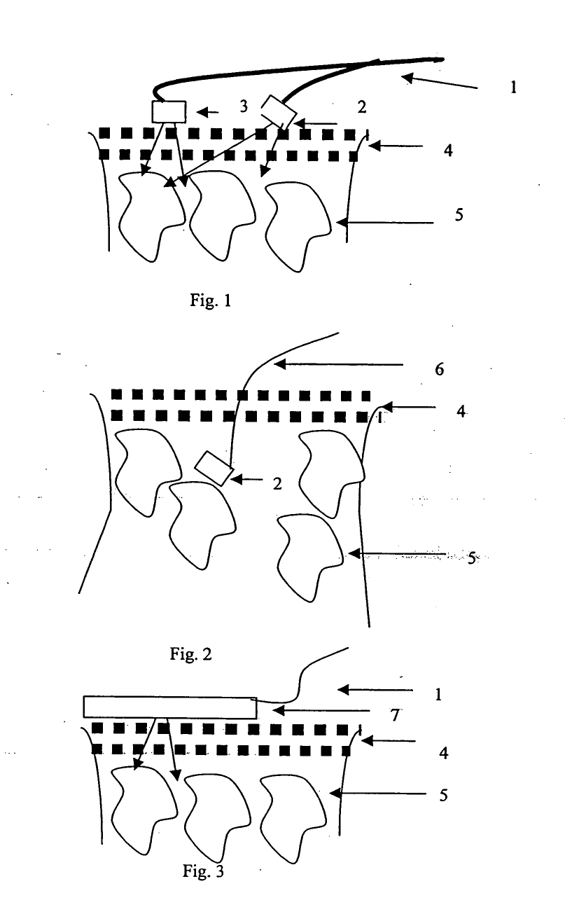

C. Following the disclosure of Example 1A and 1 B above, the PDT light source is an externally positioned light source connected (1) to a power source and directed at the site to be treated. The light source may be a laser diode, light emitting diode (3) or other electroluminescent device. The light source may be angled (2) or placed perpendicular (3) to the skin layer (4) and the light beam is focused so as to direct the light through the skin or membrane of the mammalian subject being treated to cause photoactivation of the photosensitizer agent bound to the adipocytes (5) of the adipose tissue. See FIG. 1.

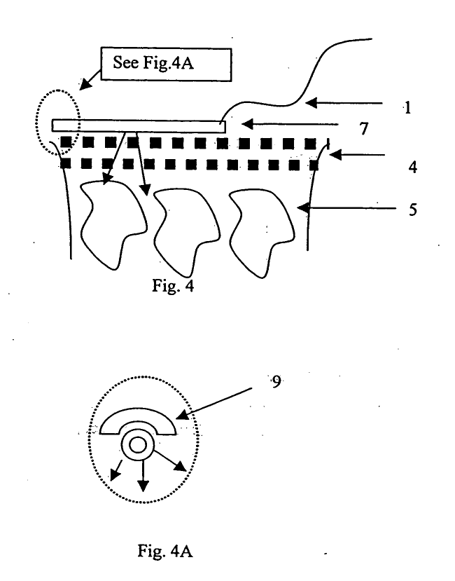

Alternatively, the light source could comprise a strip or panel of light emitting diodes (LEDs) (7), which are then arrayed on the skin or the membrane overlying the site to be treated in the mammalian subject. See FIG. 3. The light source could also comprise an optical fiber diffuser (8), which is placed over the skin or the membrane overlying the site to be treated in the mammalian subject. Such diffuser may further comprise a mirrored surface (9) directing the light beam to the target area. See FIG. 4.

D. As is apparent to one of ordinary skill in the art, the methods and compositions described above have various applications. For example, a small area of adipose tissue in a mammalian subject may be treated by utilizing a patch composed of LEDs or a mat of woven optical fibers wherein the light source patch or mat is placed over the skin or the tissue overlying the site to be treated. Furthermore, the patch or mat could also contain pharmaceutical compositions or the photosensitizing agent, which is then delivered by liposomal, transdermal or ionophoretic techniques.

### EXAMPLE 2

### Transillumination Photodynamic Therapy of Adipose Tissue

Following the method of Example 1A, a conjugate is formed between NPe⁶ and monoclonal antibody to lipoprotein lipase. Such conjugate is delivered in a manner disclosed in Example 1A. An internal light source is surgically provided by a minimally invasive procedure. The LED (2) is connected to an optical fiber (6) and surgically inserted underneath the subcutaneous layer of tissue (4). For example, Chen et al., U.S. Pat. No. 5,766,234, teach the implantation of a fiber optic fiber with an LED light source for photodynamic therapy at a local site. Also, Paolini et al., U.S. Pat. No. 5,954,710, teach a device and method of removing subcutaneous adipose layers using a laser light source connected to an optical fiber conveying means and a hollow needle for guiding the fiber, said fiber ending in the vicinity of the end of the needle.

This invention has been described by a direct description and by examples. As noted above, the examples are meant to be only examples and not to limit the invention in any meaningful way. Additionally, one having ordinary skill in the art to which this invention pertains in reviewing the specification and claims which follow would appreciate that there are equivalents to those claimed aspects of the invention. The inventors intend to encompass those equivalents within the reasonable scope of the claimed invention.

Citation of the above documents is not intended as an admission that any of the foregoing is pertinent prior art. All statements as to the date or representation as to the contents of these documents are based on the information available to the applicants and do not constitute any admission as to the correctness of the dates or contents of these documents. Further, all documents referred to throughout this application are incorporated in their entirety by reference herein.

1. A method for photodynamic therapy for the reduction of adipose tissue or adipocytes in a mammalian subject comprising:
administering to the subject a therapeutically effective amount of a photosensitizing agent or a photosensitizing agent delivery system or a prodrug, wherein said photosensitizing agent or said photosensitizing agent delivery system or said prodrug selectively localizes in the adipose tissue or the adipocytes;
irradiating at least a portion of the subject with light at a wavelength absorbed by said photosensitizing agent or if said prodrug, by a prodrug product thereof, wherein said light is provided by a light source; and wherein said irradiation is administered at a relatively low fluence rate that results in the activation of said photosensitizing agent or said prodrug product; and
wherein said PDT drug is cleared from the skin and subcutaneous tissues of the subject prior to said irradiation.

2. The method of claim 1, wherein said light source is selected from the group consisting of one or a plurality of: laser diodes; light emitting diodes;
electroluminescent light sources; incandescent light sources; cold cathode fluorescent light sources; organic polymer light sources; or inorganic light sources.

3. The method of claim 1 or claim 2, wherein said light source is external to the skin layer and the light beam is directed through the skin to the adipose tissue or the adipocytes.

4. The method of claim 2, wherein said laser diode is coupled to an optical fiber, and wherein said optical fiber directs said light to the adipose tissue or the adipocytes.

5. The method of claim 2, wherein said light emitting diode is a light emitting diode strip, and wherein said light emitting diode strip is placed external to the skin layer and overlying the adipose tissue or the adipocytes.

6. The method of claim 4, wherein said optical fiber diffuses said light when placed over the adipose tissue or the adipocytes.

7. The method of claim 4 or claim 6, wherein said light source is a mat comprising a plurality of said optical fiber.

8. The method of claim 1, wherein said photosensitizing agent is selected from the group consisting of indocyanine green, methylene blue, toluidine blue, delta-aminolevulinic acid, protoporphyrin, bacteriochlorins, phthalocyanines, porphyrins, texaphyrins, merocyanines, psoralens, pyropheophorbides, chlorins, purpurins, and any other agent that absorbs light in a range of 500 nm-1100 nm.

9. The method of claim 8, wherein said photosensitizing agent is a mono-, di- or polyamide aminodicarboxylic acid derivative of a cyclic or non-cyclic tetrapyrrole.

10. The method of claim 1, wherein said photosensitizing agent is mono-L-aspartyl chlorin e6 (NPe6).

11. The method of claim 1, wherein said wavelength is from about 500 nm to about 1100 nm.

12. The method of claim 1, wherein said wavelength is greater than about 700 nm.

13. The method of claims 1, wherein said light results in a single photon absorption mode by the photosensitizing agent.

14. The method of claim 8, wherein said photosensitizing agent is conjugated to an adipose-tissue specific ligand within a complex, wherein said ligand localizes in the adipose tissue or to the adipocytes.

15. The method of claim 14, wherein said ligand is selected from the group, an adipocyte antigen consisting of, an adipocyte cell receptor, and other adipocyte cellular surface component.

16. The method of claim 15, wherein said antigen is lipoprotein lipase.

17. The method of claim 14, wherein said complex is administered systemically or locally.

18. The method of claim 17, wherein said complex is formulated for administration orally, topically, intravenously or by any percutaneous route of injection.

19. The method of claim 17, wherein local administration is followed by a method to allow the complex to permeate the skin and into the subcutaneous adipose tissue.

20. The method of claim 8, wherein said light source is inserted internal to the skin layer of the subject.

21. The method of claim 1, wherein the reduction of the adipose tissue or the adipocytes occurs by apoptosis of the adipocytes.

22. An apparatus for transcutaneous photodynamic therapy of adipose tissue or adipocytes in a mammalian subject comprising a light source that is external to the subject and is at least one light source selected from the group consisting of laser diodes, light emitting diodes, electroluminescent light sources, incandescent light sources, cold cathode fluorescent light sources, organic polymer light sources, and inorganic light sources.

23. The apparatus of claim 22, wherein said light source is at least one laser diode coupled to an optical fiber which directs said light to the adipose tissue or the adipocytes.

24. The apparatus of claim 22, wherein said diode is a light emitting diode strip, and wherein said light emitting diode strip may be placed over the skin to contour the adipose tissue to be treated.
